(19) United States
(12) Patent Application Publication
(10) Pub. No.: US 2020/0206293 A1
(43) Pub. Date: Jul. 2, 2020

(54) METHOD FOR ENHANCING NITRIC OXIDE LEVELS IN A USER

(71) Applicant: Chinni Krishnan RAJKUMAR, Chennai, Tamil Nadu (IN)

(72) Inventor: Chinni Krishnan RAJKUMAR, Chennai, Tamil Nadu (IN)

(21) Appl. No.: 16/632,367

(22) PCT Filed: Sep. 19, 2019
(86) PCT No.: PCT/IB2018/057217

§ 371 Date: Jan. 19, 2020

Related U.S. Application Data
(60) Provisional application No. 62/535,079, filed on Jul. 20, 2017.

(51) Int. Cl. A61K 36/68; A23L 33/105; A61K 9/00

(52) U.S. Cl. CPC A61K 36/68; A61K 9/0053; A23L 33/105

(57) ABSTRACT

A method for enhancing the nitric oxide levels in a user. Nitric oxide can provide many benefits to a user including, but not limited to, improved sleeping quality, regulation of blood pressure, and strengthening of the immune system. A quantity of bacopa monnieri and a quantity of consumable produce is heterogeneously combined together into a supplement mixture. A daily dosage of the supplement mixture is administered to a user daily in order enhance the nitric oxide levels of the user. The enhancement of nitric oxide levels provides many benefits as mentioned previously and additional benefits such as, but not limited to, increasing brain function and improving memory.

### TECHNICAL FIELD

The present invention relates generally to food supplements. More specifically, the present invention is a method for enhancing nitric oxides levels in a user through a supplement mixture.

### BACKGROUND ART

Nitric oxide has many benefits to the human body including, but not limited to, improved sleeping quality, regulation of blood pressure, and strengthening of the immune system. However, there are no simple, natural, and effective solutions currently available. In more detail, current methods for enhancing nitric oxide levels involve the use of synthetic chemicals or pharmaceuticals.

It is therefore an objective of the present invention to introduce a supplement that users can utilize to overcome such problems. The present invention overcomes these problems by offering a simple supplement which can be consumed in multiple forms including, but not limited to, a beverage, cereal, or other food. The present invention is the only herbal beverage/cereal formulation/food supplement in the world which enhances the body's nitric oxide through food.

### DETAIL DESCRIPTIONS OF THE INVENTION

All illustrations of the drawings are for the purpose of describing selected versions of the present invention and are not intended to limit the scope of the present invention.

The present invention is a method for enhancing nitric oxide levels in a user. Nitric oxide has many benefits to the human body including, but not limited to, improved sleeping quality, regulation of blood pressure, and strengthening of the immune system. An increase of nitric oxide in a body causes an increase in cyclic guanosine monophosphate (GMP) which further causes an activation of intracellular protein kinases. The activation of intracellular protein kinases is the cause for smooth muscle relaxation, platelet function, and other benefits. Furthermore, nitric oxide is a “secondary messenger molecule” which carries information from one brain cell to another brain cell, which enhances the energy to the brain to function with high intelligence quotient (IQ) capacity. In the blood vessels, the present invention dilates by increasing the “endothelial relaxant factor” (EFDF), thereby increasing the blood circulation. Overall, daily consumption of the present invention will increase the nitric oxide levels in a user and keep the body and mind healthy.

In order to execute the steps of the present invention, the present invention requires a quantity of bacopa monnieri and a quantity of consumable product. Bacopa monnieri is a natural herb which is effective in providing nitric oxide to a user when consumed by the user. Moreover, bacopa monnieri is used as a nerve tonic for the improvement in memory of a user. The consumable product may be any type of consumable such as, but not limited, a beverage or cereal. The consumable product makes the present invention desirable to consume. In accordance to the preferred embodiment of the present invention, the quantity of bacopa monnieri and the quantity of consumable product are heterogeneously combined into a supplement mixture. The supplement mixture can contain varying quantities of bacopa monnieri and consumable product. The varying quantities of bacopa monnieri and consumable product is dependent on the attributes of the user.

Once the supplement mixture is formed, a daily dosage of the supplement mixture is administered to the user. While the daily dosage is able to be administered through a number of the methods to the user, the daily dosage is preferred to be administered orally to the user. The daily dosage is dependent on the attributes of the user such as, but not limited to, the age of the user. Moreover, depending on the activities that the user is associated with, the daily dosage may vary in order to increase the performance of the user in his or her respected activity.

In a first embodiment of the present invention, the supplement mixture is a liquid mixture. The liquid mixture is preferably a beverage that is easily consumable. The daily dosage of the liquid mixture is preferably between 120 to 250 millilitres. For the liquid mixture, this quantity of the daily dosage is enough to enhance the nitric oxide levels in a user.

In a second embodiment of the present invention, the supplement mixture is a solid mixture. The solid mixture can be any type of consumable that is a solid such as, but not limited to, a cereal or capsule. The daily dosage of the solid mixture is preferably between 120 to 250 milligrams. For the solid mixture, this quantity of the daily dosage is enough to enhance the nitric oxide levels in a user.

In order to scientifically confirm that the nitric oxide levels have been enhanced in a user, the present invention requires a saliva test strip. The nitric oxide level of a user is measured through the saliva test strip. The enhancement of the user's nitric oxide level can be measured in 15 to 30 seconds through the saliva test strip. In order to receive the best results, the nitric oxide level of the user is measured through the saliva test strip within 30 to 45 minutes after administering the daily dosage. A reference guide for the results of saliva test strip is provided to verify the enhancement of the user's nitric oxide levels. In the preferred embodiment of the present invention, the saliva test strip should show a darkening of pink color which indicates an increase in nitric oxide levels in a user.

Phase I Clinical Trial with Bacosides

This study was carried out in two parts—single dose and multiple dose tolerance study in healthy male volunteers.

Single Dose Study:

Phase I single dose tolerance study was undertaken at B.R.D. Medical College, Gorakhpur. Thirty-one normal male volunteers who consented to undergo the trial as per approved protocol completed the study. The trial was double blind placebo controlled and non-crossover. The dose of bacosides A&B ranged from 20 mg to 300 mg. Six volunteers received placebo while 4 each were given 20 mg, 50 mg, 75 mg, 100 mg, 150 mg, 200 mg and 1 volunteer received 300 mg bacosides in gradually escalated manner.

In all cases Bacosides A&B was well tolerated. Recording of vital parameters (pulse, blood pressure and general condition) at 0, 1, 2, 3, 4, 6, and 24 hours post medication were normal limits. The hematological parameters (Hb, TLC, DLC, PCV, ESR), biochemical parameters (blood sugar, blood urea, creatinine, bilirubin, SGOT, SGPT, AP, serum proteins, total cholesterol and triglycerides) and electrocardiogram were within normal range in +24 hours of post medication.

Multiple Dose Study:

Twenty healthy male volunteers who executed the written informed consent, completed the study as per approved protocol. Initially the first group of 10 volunteers was give Bacoside (100 mg) orally once a day for 4 weeks. Subsequently, the second group of 10 healthy male subjects received Bacoside (200 mg) orally for 4 weeks.

Each volunteer was subjected to a detailed history, clinical examination, and laboratory investigations before the administration of first dose of Bacosides with the aim of having initial baseline pretherapy values of each parameters. Subsequently all the volunteers were closely monitored during the trail. Every volunteer was examined for vital parameters (blood pressure and pulse) and subjective experiences every week during the 4-week study. In each case all baseline clinical and laboratory investigations were repeated at the end of 4-week trial. A critical appraisal of these observations revealed that no drug related abnormalities could be detected in clinical and laboratory parameters. Thus, it can be concluded that Bacosides A&B with 100 mg and 200 mg doses given orally each day for 4 weeks is well tolerated and does not produce any undesirable side effects or abnormality on these dose levels (5).

Phase II Clinical Trial with Bacosides

Materials and Methods:

The present study was carried out in the Department of Pediatric, B.R.D. Medical College, Gorakhpur, Lucknow. The period of study was one year four months starting from August 1997 to November 1998. The aim of the study was to evaluate memory enhancing properties of Memory plus in children with Attention Deficit Hyperactivity Disorder (ADHD).

SUBJECTS: The study included 36 children of ADHD fulfilling the criteria of Diagnostic and Statistical Manual, 4th Edition (DSM IV) of American Psychiatric Association Committee.

Sample Selection:

The children between the age of 6 to 12 years were selected from Outpatient Department (OPD) of Pediatrics, B.R.D. Medical College, Gorakhpur. In addition, the children from nearby school were also screened and children found with ADHD were selected for the study. Each subject was evaluated for intelligence quotient (IQ) wherever possible. Those children who were found having visual, hearing impairment, seizure disorder, acute/chronic diarrhea were excluded.

Ethics:

Prior to each study parents/guardian of each child was explained in such a manner that he/she could completely understand that chills had been treated with a new drug. Written informed consent was obtained from parent/guardian of each child enrolled in the study.

Material:

Capsules of identical color and size containing 50 mg Memory Plus powder and equal amount of placebo, were used.

Design:

Double blind, randomized, placebo-controlled design was used.

Statistical Method:

1. We have applied paired “t” Test to test the significance between the periods of visits i.e. from initial pre-drug test score to subsequent values observed at 4 weekly intervals (+4, +8, +12, &+16 weeks).

2. To test the efficacy of Memory Plus as compared with Placebo, first we have subtracted the initial test score from subsequent week's values then nonparametric test, “u’ Test (Mann Whitney) was applied.

Results:

The present study was carried out in the Department of Pediatrics, Baba Raghav Das Medical College, Gorakhpur, Lucknow, from August 1997 to November 1998. The study was designed to evaluate the efficacy and tolerability of Memory Plus in children suffering from Attention Deficit Hyperactivity Disorder. The treatment was assigned as per random allocation into two groups receiving Memory Plus and Placebo. The following conclusions were drawn from the present double-blind controlled study.

1. Thirty-six patients were selected for the study period as per Diagnostic & Statistical Manual (DSMIV) OF American Psychiatric Association Committee criteria, 19 in Memory Plus and 17 in Placebo group.

2. In this FIGURE included drop out were, one in Memory Plus and six in Placebo group.

3. The mean age was 8.32 years and 9.3 years in Memory Plus and Placebo group respectively.

4. Maximum number of patients were in age group of 6-8 years and 10-12 years in placebo group as well as in Memory Plus group.

5. The male:female ratio was 5.3:1 and 3.3:1 in Memory Plus and placebo group respectively.

6. As per educational distribution data, most of the children 36.8% and 41.2% of Memory Plus and Placebo group respectively were studying in Class First whereas the minimum number of children 5.3% were in class Third in Memory Plus and 5.9% in Fifth Class from Placebo group.

7. So far, the history of past illness and possible etiological factor is concerned, perinatal asphyxia and viral encephalitis dominated in Placebo group as 5(29.5%) and 4(23.53%) cases respectively, whereas in Memory Plus high-grade fever 4(21.1%) and viral encephalitis 4(21.1%) were the two equal predominating possible etiological factors. There was no reported history of past illness in 7(36.8%) in Memory Plus and 5(29.4%) cases in placebo group.

8. Following tests were applied to assess memory enhancing properties and effect of Memory Plus in ADHD during study period.

(i) Personal information

(ii) Mental control

(iii) Sentence repetition

(iv) Logical memory

(v) Word recall (meaningful)

(vi) Digit span test

(vii) Word recall (non-meaningful)

(viii) Delayed response learning

(ix) Picture recall

(x) Paired associate learning

9. A significant improvement was observed in scoring of mental control, sentence repetition word recall (meaningful) picture recall, paired associate learning right from the 4th week duration of treatment trail and from 8th week onwards in personal information, digit span test, word recall (non-meaningful) and logical memory. However, there was no significant change on delayed learning. (Tables I to 11).

10. There was no significant improvement in scores in any of these tests in placebo group except for mental control, sentence repetition and picture recall, where there was an improvement at 12 weeks of study. Further, in memory plus group there was a highly significant effect on improvement in all the tests except delayed response learning from 8th week of clinical trial.

11. The effect was maintained even after drug withdrawal from beginning of 13th week to 16th week, which supports the withdrawal free effect of Memory Plus.

12. A pre and post drug monitoring of clinical hematological and biochemical parameters did not suggest any drug related abnormality in both the treatment groups. This indicates the excellent tolerability and safety of the drug, in the subjects of study.

13. In contrast to Placebo group some of the patients of memory plus group, 4(22.2%) reported improvement in their appetite at the end of 4 weeks, of the trail and 13 (72.2%) cases reported improvement in their concentration which was confirmed objectively in the memory test.

14. The data observed from the present study have confirmed the efficacy of Memory Plus in the treatment of ADHD.

15. The sample size of patients for the study was limited to only 36 instead of required 60 patients because of certain reasons beyond our control. However, the present study strongly supports the safety and efficacy tolerance of Memory Plus and it facilitates the learning process through enhancement of memory.

TABLE 11
Distribution of comparative change in Test score
from 0 week to 12 weeks in Memory Plus and Placebo group.
(Values expressed as mean _+ SD (n)

| | Memory Plus | Placebo | Signifi- |
|---|---|---|---|
| Test | Group | Group | cance |
| Personal | 0.8 + _0.80 (18) | 0.3 + _0.5 (11) | N.S. |
| Information | | | |
| Mental Control | 1.80 + _2.5 (18) | 1.20 + _1.6 (18) | N.S. |
| Sentence | 2.4 + _1.8 (18) | 0.70 + _1.0 (11) | ** |
| Repetition | | | |
| Logical Memory | 3.2 + _2.3 (18) | 0.5 + _I.I (11) | ** |
| Word Recall | 2.4 + _2.1 (18) | 2.10 + _3.5 (11) | N.S. |
| (Meaningful) | | | |
| Digit Span Test | 2.7 + _1.9 (18) | 2.0 + _3.4 (11) | N.S. |
| Word Recall | 1.5 + _1.9 (18) | 0.70 + _0.9 (11) | N.S. |
| (Non-meaningful) | | | |
| Picture Recall | 1.05 + _1.0 (18) | 0.40 + _0.5 (11) | N.S. |
| Delayed | 0.47 + _I.I (18) | 0.40 + _0.70 (11) | N.S. |
| Response | | | |
| Learning | | | |
| Paired | 4.9 + _4.0 (18) | 1.4 + _2.1 (11) | ** |
| Associate | | | |
| Learning | | | |
| Total Memory | 21.7 + _12.5 (18) | 6.1 + _8.3 (11) | ** |
| Test score | | | |

** Highly Significant (P < 0.01),
N.S.—Not Significant (P > 0.05)

The above table shows the highly significant change in Sentence Repetition, Logical Memory and Paired Associate Learning Test scores and Total score values of Memory Plus group as compared to Placebo group. However, in other Test score of the study, there was the tendency of increasing values as compared to Placebo group.

Double Blind Clinical Study of Memory Plus/Gingko Biloba/Placebo in Elderly Subjects Suffering from Age Associated Memory Impairment:

A double-blind placebo controlled randomized study was performed in elderly individuals (above the age of 45 years). Patients with subjective evidence of age associated memory loss and who satisfied the inclusion evidence of age associated memory loss and who satisfied the inclusion criteria (complaints of memory loss in everyday activities, a cut off score of <6 on Weschler Memory Scale, score of >24 on Mini Mental State Examination and a score of >9 on Vocabulary subset of Wechsler Adult Intelligence Scale) were recruited for this study and were randomly assigned to receive either Memory plus (125 mg capsule) or (Gingko biloba (60 mg capsule) or placebo (identical capsule) twice a day for 12 weeks. Assessment was done on day 0, 4th, 8th, 12th, and 16th week (i.e. 4 weeks after stopping therapy). This included clinical history, laboratory investigation and psychometric tests i.e. Wechsler's Memory Scale (WMS) & Mini Mental State Examination (MMSE).

A total of thirty-two patients were included in the study. Out of these 11 received memory plus, 12 received Gingko biloba and 9 were given placebo. Out of 11 patients (7 males+4 females) who received Memory plus, 2 patients dropped out and 9 completed the trail. The mean age of these patients was 62.0+10.4 years.

A detailed analysis of haematological and biochemical parameters at 0, 4, 8 and 12 and 16 weeks of drug administration (Memory plus/Gingko biloba or placebo did not reveal any significant deviation from the baseline (0 week). This establishes the safety of memory plus (125 mg BD) in nine elderly subjects after 12 weeks of drug administration.

Although the invention has been explained in relation to its preferred embodiment, it is to be understood that many other possible modifications and variations can be made without departing from the spirit and scope of the invention as hereinafter claimed.

1. A method for enhancing nitric oxide levels in a user, the method comprises the steps of:
providing a quantity of bacopa monnieri and a quantity of consumable product;
heterogeneously combining the quantity of bacopa monnieri and the quantity of consumable product into a supplement mixture; and
administering a daily dosage of the supplement mixture to the user.

2. The method for enhancing nitric oxide levels in a user, the method as claimed in claim 1, wherein the daily dosage is administered orally to the user.

3. The method for enhancing nitric oxide levels, as claimed in claim 1, wherein the supplement mixture is a liquid mixture.

4. The method for enhancing nitric oxide levels, as claimed in claim 3, wherein the daily dosage is between 125 to 250 millilitres.

5. The method for enhancing nitric oxide levels, as claimed in claim 1, wherein the supplement mixture is a solid mixture.

6. The method for enhancing nitric oxide levels, as claimed in claim 5, wherein the daily dosage is between 125 to 250 milligrams.

7. The method for enhancing nitric oxide levels, as claimed in claim 1, comprises: providing a saliva test strip; and measuring the nitric oxide level of a user through the saliva test strip.

8. The method for enhancing nitric oxide levels, as claimed in claim 7, comprises: measuring the nitric oxide level of the user through the saliva test strip within 30 to 45 minutes after administering the daily dosage.
